(19) United States
(12) Patent Application Publication
(10) Pub. No.: US 2008/0241967 A1
(43) Pub. Date: Oct. 2, 2008

(54) Radioimmunoassay using nanoparticle-antibody conjugates

(76) Inventors: Feng Chen, Central, SC (US); A. B. Bodine, Clemson, SC (US); Paul L. Dawson, Clemson, SC (US); Thomas R. Scott, Central, SC (US); Xi Wang, Central, SC (US)

Correspondence Address: LEIGH P. GREGORY, PO BOX 168, CLEMSON, SC, 29633-0168, US

(21) Appl. No.: 12/069,136

(22) Filed: Feb. 6, 2008

Related U.S. Application Data
(62) Division of application No. 11/244,567, filed on Oct. 6, 2005.

(51) Int. Cl. G01N 33/536

(52) U.S. Cl. 436/542

(57) ABSTRACT

A radioimmunoassay method for determining the quantity of an analyte of interest in a sample is disclosed. The analyte of interest may be an antigen or other chemical entity. A known antibody to the antigen or other entity is employed and is conjugated to a functionalized nanoparticle. Because of the high surface area presented by the present nanoparticle—antibody conjugates, the present radioimmunoassay method is particularly suited for the qualitative and quantitative analysis of low molecular weight chemicals.

### CROSS-REFERENCE TO COPENDING APPLICATIONS

The present application is a divisional application of U.S. Ser. No. 11/244,567, filed Oct. 6, 2005.

### FIELD OF THE INVENTION

The present invention is directed to radioimmunoassay techniques for detecting analytes and, specifically, is directed to radioimmunoassay methods using nanoparticle-antibody conjugates.

### BACKGROUND OF THE INVENTION

Typically, radioimmunoassays are based on the reaction between an antibody and an antigen whose concentration has to be quantified. There are several ways to quantify the antigen concentration but the most frequently used method is the indirect assay. In this assay a known quantity of radioactively labeled antigen is mixed with a dilution series of “cold” antigen. The dilution series is brought to reaction with a fixed amount of antibody specific against the antigen. Since cold and radioactively labeled antigens compete with each other for the antibody binding sites, a high concentration of antigen will result in little radioactive antigen bound to the antibody and vice versa. After each reaction the antibody-bound antigen is separated from the free antigen in the supernatant fluid based on size or magnetic function. The radioactivity of each fraction is then measured. The serially diluted probes yield points on a curve relating radioactive counts to the concentration of cold antigen: a so-called (cold) reference curve. Using this reference curve, an unknown quantity of antigen in a probe can be quantified by performing the same reaction. Identification of the radioactive counts in the supernatant fluid and use of the reference curve yields the unknown antigen concentration.

In prior radioimmunoassay methods the antibody has been fixed on an immobile substrate or on large, polymeric beads. Although these methods have been adequate for detecting and quantifying the presence of antigens at certain levels, because of the limited surface areas of these substrates sensitivity has been limited.

### SUMMARY OF THE INVENTION

Thus, in one aspect the present invention is directed to a method for detecting the quantity of an analyte of interest in a sample comprising the steps of: providing a known antibody to the analyte; providing a nanoparticle; functionalizing the nanoparticle; conjugating the antibody to the functionalized nanoparticle; introducing a quantity of radioactively labeled analyte to a quantity of the conjugate, thereby binding the radioactively labeled analyte to the antibody conjugate; introducing the fluid sample of the analyte of interest, thereby displacing a portion of the radioactively labeled analyte; separating the analyte bound antibody conjugate from the supernatant fluid of the sample; measuring the radioactivity of each of the analyte bound antibody conjugate and the supernatant fluid; and comparing the quantity of displaced radioactively labeled analyte to a known curve.

In another aspect the present invention is directed to a method for determining the quantity of an analyte of interest in a sample, wherein an antibody is employed to bind the analyte and wherein the antibody is conjugated to a functionalized nanoparticle.

### DETAILED DESCRIPTION OF THE PREFERRED EMBODIMENTS

Basically, the present invention is directed to a radioimmunoassay method having increased sensitivity as compared to known radioimmunoassays. The present method employs nanoparticle-antibody conjugates rather than conventional plates, columns or beads for carrying the antibodies. Thus, the antibodies move freely in solution as a capturer and concentrator in light of presenting an increased surface area to the analyte of interest.

The present method may be employed for any known antibody—antigen reaction and is especially suited to the quantitative determination of small amounts of low molecular weight chemicals, such as bioterrorism toxins, environmental contaminants such as shellfish poisoning toxins, mycotoxins, aflatoxins, agricultural crop drugs (insecticides, fungicides, herbicides), athletic doping drugs, medicinal drugs, etc. The present conjugates also may be employed in the design of drug missiles for a variety of medicinal therapies.

By the present inventive method nanoparticles are functionalized, preferably biofunctionalized, by exposure to a functionalizing agent capable of binding the desired antibody. A particularly useful functionalizing agent is bovine serum albumin although a variety of agents may be employed.

As with all forms of radioimmunoassay, the antibody employed depends on the antigen or chemical entity, broadly the analyte of interest, being detected. Generally, the present invention is directed to a method for detecting the quantity of an analyte of interest in a fluid sample which requires providing a known antibody to the analyte; conjugating the antibody to a functionalized nanoparticle; and then introducing a quantity of radioactively labeled analyte to a quantity of the antibody conjugate, thereby binding the radioactively labeled analyte to the antibody conjugate. Thereafter, the fluid sample of the analyte of interest is introduced, displacing a portion of the radioactively labeled analyte. The analyte bound antibody conjugate is separated from the supernatant fluid of the sample either by centrifugation or magnetically, if the nanoparticle being used displays magnetic properties. Then the radioactivity of each fraction is measured. The results are compared to a known curve for the given antibody—analyte reaction. Such curve may be formed by sequentially performing these steps with varying, known quantities of the non-radioactively labeled analyte and plotting the results. Then the results for an analyte sample of unknown quantity may be compared to the curve.

### Example 1

A nanoparticle—PCB antibody conjugate was prepared for use in radioimmunoassay of PCB. Nanoparticle-bovine serum albumin (NP-BSA) conjugates were obtained from Clemson University. A 750 μl suspension of NP-BSA was centrifuged at maximum speed for 5 minutes and the pellet was collected. The pellet was washed with a buffer solution and then dissolved in 300 μl of 0.1 M MEW buffer at a pH of 6.3. Five aliquots of 60 μl each were taken into each of five tubes. The NP-BSA conjugates were reacted with varying amounts of PCB antibody in the presence of EDAC as follows in Table 1.

TABLE 1

| Tube No. | 1 | | 2 | | 3 | | 4 | | 5 | |
|---|---|---|---|---|---|---|---|---|---|---|
| NP-BSA | 60 | μl | 60 | μl | 60 | μl | 60 | μl | 60 | μl |
| Antibody | 9.25 | μl | 19.5 | μl | 27.75 | μl | 37 | μl | 46.25 | μl |
| EDAC | 5 | μl | 7.5 | μl | 10 | μl | 12.5 | μl | 15 | μl |
| Rx time | 3 | hrs. | 3 | hrs. | 3 | hrs. | 3 | hrs. | 3 | hrs. |

After the incubation time the conjugates were centrifuged for five minutes, collected, and washed two times with PBS.

The nanoparticle-antibody conjugates of Table 1 were then diluted to 1000 μl with PBS. Varying amounts of C¹⁴ PCB in μCi levels were added and the solutions were shaken gently for one hour at 25° C. Thereafter, the PCB bound antibody conjugates were separated by centrifuging for five minutes. The pellets were taken up in 20 μl PBS each and then loaded into a 4 ml Aquasol-2 scintillation cocktail solutions obtained from Sigma. Radioactivity was measured with a liquid scintillation counter using a C¹⁴ program. The sample preparation conditions are set forth in Table 2 and the results are set forth in Table 3, both below. In Table 3, higher counts correspond to increasing amounts of PCB.

TABLE 2

| Tube | C¹⁴ PCB in μCi | Tube | Supernatant from tubes 1-5 |
|---|---|---|---|
| 1 | 0.2 | 6 | SN 1 |
| 2 | 0.3 | 7 | SN 2 |
| 3 | 0.4 | 8 | SN 3 |
| 4 | 0 as control | 9 | SN 4 as control |
| 5 | 0 as control | 10 | SN 5 as control |

TABLE 3

| PCB (μCi) | Count |
|---|---|
| 0 | 14 |
| 0.2 | 2134 |
| 0.3 | 2226 |
| 0.4 | 2681 |

Table 4, below, shows the results of exposing varying amounts of the antibody conjugate to a constant quantity, 0.1 μCi, of radioactive PCB.

TABLE 4

| Antibody - Nanoparticle Conjugate (ml) | Count |
|---|---|
| 0 | 15 |
| 0.5 | 6521 |
| 1.0 | 31,693 |

TABLE 5

| | BSA | EDAC | Antibody | PCB | |
|---|---|---|---|---|---|
| | [M] | [M] | [M] | [M] | Count |
| | (10⁻¹⁰) | (10⁻⁷) | (10⁻¹⁰) | (10⁻⁹) | (cpm) |
| Exp. | 7.4 | 2.6 | 7.2 | 16 | 2,134 |
| 1 | 7.4 | 2.6 | 14 | 24 | 2,226 |
| | 7.4 | 2.6 | 21 | 32 | 2,681 |
| Exp. | 25 | 2.6 | 24 | 7.9 | 6,521 |
| 2 | 50 | 2.6 | 48 | 7.9 | 31,693 |

Assuming a 1:1 chemical to antibody reaction, experiments 1 and 2 in Table 5, above, should yield counts in the magnitude of 105. However, actual counts were much lower. Counts in Table 3 were relatively low compared to those in Table 4 because low concentration of BSA and antibody precluded high number of chemical molecules binding to NP-BSA-antibody complex. It appears that the low concentration of NP-BSA-antibody was the bottleneck, thus even though PCB concentrations in experiment 1 (Table 5) were much higher, most did not bind to antibody and were washed out prior to scintillation counting. Higher concentrations of NP-BSA were used in experiment 2; the increased number of conjugates resulted in higher activity with PCB and higher counts. Counts for experiment 2 (Table 5) are also lower than predicted, possibly due to a shielding effect of conglomerated particles which haven't dispersed evenly in the Aquasol-2 cocktail. Nevertheless, this method has demonstrated that the detecting sensitivity is at least 10 times higher than traditional chromatographic methods. Also, this method is simpler, more economical, requires less laboratory expertise, suitable for the field test for rapid response, and could therefore be used for high through-put screening.

While the disclosed process has been described according to its preferred embodiments, those of ordinary skill in the art will understand that numerous other embodiments have been enabled by the foregoing disclosure. Accordingly, the foregoing embodiments are merely exemplary of the present invention. Modifications, omissions, substitutions and rearrangements may be made to the foregoing embodiments without departing from the invention as set forth in the appended claims.

1. A method for detecting the quantity of an analyte of interest in a fluid sample comprising the steps of:
a) providing a known antibody to the analyte;
b) providing a nanoparticle;
c) functionalizing the nanoparticle;
d) conjugating the antibody to the functionalized nanoparticle;
e) introducing a quantity of radioactively labeled analyte to a quantity of the antibody conjugate, thereby binding the radioactively labeled analyte to the antibody conjugate;
f) introducing the fluid sample of the analyte of interest, thereby displacing a portion of the radioactively labeled analyte;
g) magnetically separating the analyte bound antibody conjugate from the supernatant fluid of the sample;
h) measuring the radioactivity of each of the analyte bound antibody conjugate and the supernatant fluid; and
i) comparing the quantity of displaced radioactively labeled analyte to a known curve.

2. The method set forth in claim 1 wherein the analyte of interest comprises a low molecular weight chemical.

3. The method set forth in claim 1 wherein the analyte of interest comprises an antigen.

4. (canceled)

5. (canceled)
